SEQUENCE LISTING (9 sequences)

SEQ ID NO: 1 - DNA, 29 bp, Artificial
  synthetic
atacccgaagtcaattagtgacgtcgttg

SEQ ID NO: 2 - DNA, 29 bp, Artificial
  synthetic
tggactactgttcatgacgaattcctttg

SEQ ID NO: 3 - DNA, 20 bp, Artificial
  synthetic
gccctttccaacagattcaa

SEQ ID NO: 4 - DNA, 21 bp, Artificial
  synthetic
gatattggcgcggctcaatca

SEQ ID NO: 5 - DNA, 22 bp, Artificial
  synthetic
aggcgacggagtttactggaag

SEQ ID NO: 6 - DNA, 23 bp, Artificial
  synthetic
caaaagccagagcaaacctctga

SEQ ID NO: 7 - DNA, 36 bp, Arabidopsis thaliana
cgcaagggatattggcgcggctcaatcaccatctca

SEQ ID NO: 8 - DNA, 21 bp, Arabidopsis thaliana
ctataaccgcgccgagttagt

SEQ ID NO: 9 - DNA, 37 bp, Arabidopsis thaliana
cggtacccggttttccttcttttttccaagcagttcg

(19) United States
(12) Patent Application Publication
(10) Pub. No.: US 2006/0252072 A1
(43) Pub. Date: Nov. 9, 2006

(54) Methods of identifying aberrant RNA or RNA targeted for cleavage by miRNA or siRNA

(76) Inventors: Pamela J. Green, Newark, DE (US); Frederic F. Souret, Newark, DE (US); James P. Kastenmayer, Germantown, MD (US)

Correspondence Address: Connolly Bove Lodge & Hutz LLP;1007 North Orange Street, P.O. Box 2207, Wilmington, DE, 19899, US

(21) Appl. No.: 11/395,423

(22) Filed: Mar. 31, 2006

Related U.S. Application Data
(60) Provisional application No. 60/666,521, filed on Mar. 31, 2005.

(51) Int. Cl. C12Q 1/68; A01H 1/00; C12N 15/82

(52) U.S. Cl. 435/6; 800/285

(57) ABSTRACT

The invention provides novel methods of identifying RNA that is targeted for cleavage by mRNA or siRNA or genes that give rise to aberrant RNA. The invention also provides the use in processes for identifying aberrant RNA or RNA targeted for cleavage by mRNA or siRNA of mutant eukaryotic cells or non-human organisms in which the gene encoding the enzyme AtXRN4 or its homologue is defective.

### REFERENCE TO UNITED STATES GOVERNMENT SUPPORT

The present invention was supported in part by a grant from the National Science Foundation, contract number 0228144. The United States Government has certain rights in the invention.

### FIELD OF THE INVENTION

The invention relates to the field of RNA degradation. More particularly, the invention relates to methods of identifying aberrant RNA or RNA targeted for cleavage by mRNA or siRNA.

### BACKGROUND OF THE INVENTION

The control of mRNA stability is an important component of gene expression that influences many aspects of growth and development. Among eukaryotes, mRNA decay mechanisms have been primarily dissected in Saccharomyces cerevisiae in which the majority of transcripts are degraded via two major pathways (Parker and Song, 2004). The deadenylation-dependent-decapping pathway involves poly(A) shortening followed by removal of the cap and 5′ to 3′ degradation of the transcript (Wilusz et al., 2001). Molecular genetic and biochemical approaches have established that Xrn1p is the 5′ to 3′ exoribonuclease (XRN) that plays a central role. Transcripts can also be degraded by a 3′ to 5′ pathway that involves an enzyme complex known as the exosome (Jacobs et al., 1998). In vitro studies support a major contribution of 3′ to 5′ exoribonucleolytic degradation in mRNA decay in mammals (Wang and Kiledjian, 2001). Similar to exosome components, XRN homologs are present in a number of other organisms, including plants (Gutierrez et al., 1999; Kastenmayer and Green, 2000) and mammals, and the proteins have been associated with complexes thought to facilitate mRNA decay (Lejeune et al., 2003; Sheth and Parker, 2003). These data are consistent with the conservation of XRN-mediated mRNA degradation in multicellular eukaryotes, although in vivo evidence is lacking.

Developments in the area of mRNA decay have emerged from studies of mRNA cleavage mediated by 21-24 nt small interfering RNAs (siRNAs) and microRNAs (mRNAs). Although absent in S. cerevisiae, these RNAs are present in plants, animals, and some fungi and function to silence genes by several mechanisms (Bartel, 2004; Cerutti, 2003). To exert their cleavage function, siRNAs are incorporated into a protein complex, termed RNA induced silencing complex (RISC) (Hannon, 2002) while mRNAs are incorporated into a similar or identical miRNP complex (Mourelatos et al., 2002; Schwarz and Zamore, 2002). Perfect or near-perfect antisense sequence complementarity between siRNAs/mRNAs and their targets directs the RISC and miRNP complex to cleave the target mRNA near the center of the paired region (Kasschau et al., 2003). How the cleaved target mRNA is subsequently degraded is unknown but cytoplasmic exoribonucleases could be involved. Interestingly, the 3′ cleavage products produced by RISC (Hannon, 2002), and the miRNP (Llave et al., 2002b), have a 5′ monophosphate which is the preferred substrate for XRN exoribonucleases (Stevens, 1979). However, the functional contributions of Xrn1p homologs in the both the general and mRNA/siRNA-mediated RNA decay pathways of multicellular eukaryotes remain to be clarified.

It has been shown that Arabidopsis produces three XRN enzymes (AtXRNs), among which AtXRN4 is the best candidate for a functional homolog of Xrnlp (Kastenmayer and Green, 2000). Yeast complementation and GFP-fusion experiments in plants indicate that AtXRN4 is localized in the cytoplasm, both in plant cells and when introduced into yeast (Kastenmayer and Green, 2000). Similar to the situation for homologs in other multicellular eukaryotes, the RNA substrates of AtXRN4 have not been identified.

Moreover, study of RNA decay is hindered by the transient presence of the transcripts themselves. Methods of studying RNA decay that facilitate the accumulation of RNA decay intermediates, or aberrant RNAs are needed.

### SUMMARY OF THE INVENTION

The invention provides novel methods of identifying RNA that is targeted for cleavage by mRNA or siRNA or genes that give rise to aberrant RNA. The invention also provides the use in processes for identifying aberrant RNA or RNA targeted for cleavage by mRNA or siRNA of mutant eukaryotic cells or non-human organisms in which the gene encoding the enzyme AtXRN4 or its homologue is defective. The use of such mutant cells or non-human organisms facilitates rapid accumulation to detectable levels of such RNA.

Thus, the invention provides method of identifying aberrant RNA or RNAs targeted for cleavage by mRNA or siRNA comprising the steps of optionally treating mutant cells or a non-human organism in which the gene encoding AtXRN4 or its homologue is defective with an agent that inhibits RNA synthesis; isolating RNA from the cells or non-human organism; and identifying aberrant RNA or RNA cleavage products in the isolated RNA by comparison with the wild type cells or non-human organism.

In one preferred embodiment of the invention, the identifying step comprises the steps of preparing microarray probes from the isolated RNA; hybridizing the microarray probes with a microarray comprised of DNA that represents gene transcripts from the same or similar type of cells or non-human organism; detecting changes in hybridization of the microarray probes from AtXRN4 mutants relative to that from the wild type; relating a changed hybridization signal from a probe with the identity of the corresponding gene represented on the microarray; and characterizing the hybridized RNA as containing RNA or an RNA cleavage product by comparison with known nucleic acid sequences selected from the group consisting of genes encoding mRNA, cDNA sequences, mRNA sequences or siRNA sequences. Preferably, the characterizing step comprises the use of Northern blot, Rapid Amplification of Complementary Ends (RACE), reverse transcriptase-polymerase chain reaction (RT-PCR) or DNA sequencing in characterizing the RNA as aberrant RNA or an RNA cleavage product.

In another preferred embodiment of the invention the identifying step comprises the steps of preparing probes corresponding to at least one gene or other nucleic acid sequence of interest; hybridizing the probes with a Northern blot that contains isolated RNA from the mutant cells or non-human organism and wild type RNA from the same or similar type of cells or non-human organism; detecting hybridization of the probes to RNA on the Northern blot; and characterizing the RNA as aberrant RNA or an RNA cleavage product by comparison with the wild type or with known nucleic acid sequences selected from the group consisting of genes encoding mRNAs, cDNA sequences, mRNA sequences or siRNA sequences. Preferably, the characterizing step comprises the use of Northern blot, oligo-directed RNaseH cleavage, Rapid Amplification of Complementary Ends (RACE), reverse transcriptase-polymerase chain reaction (RT-PCR) or DNA sequencing in characterizing the RNA as aberrant RNA or an RNA cleavage product.

A preferred mutant non-human organism for use in the methods of the invention is a mutant Arabidopsis thaliana plant wherein the gene encoding XRN4 is defective. Preferably, in plants the gene encoding AtXRN4 is disrupted by a T-DNA insert that renders the gene defective.

These and other aspects of the invention are set out in more detail in the following Detailed Description and in the appended claims.

### BRIEF DESCRIPTION OF THE DRAWINGS

FIG. 1 shows identification of AtXRN4 T-DNA insertion lines.

FIG. 1A shows a schematic representation of Arabidopsis thaliana XRN4 mutants. Mutant xrn4-5 contains a T-DNA insert in exon 18 (position 1858 in the coding region and position 5007 in the genomic sequence relative to the ATG). Mutant xrn4-4 contains a T-DNA insert in intron 12 (position 3695 in the genomic sequence). Filled circles represent stop codons.

FIG. 1B shows a RNA blot analysis of AtXRN4 mRNA accumulation using full-length AtXRN4 cDNA as probe.

FIG. 1C shows a schematic representation of two truncated AtXRN4 cDNAs, 5′ ends 1 and 2, amplified and cloned from homozygous xrn4-4 plants. AtXRN4 5′ end 1 consists of 1461 bp (486 amino acids), and AtXRN4 5′end 2 consists of 1683 bp (560 amino acids including 41 amino acids derived from intron 12 [stippled line]). The ORF of both transcripts stops in the left border of the T-DNA [LB].

FIG. 1D shows the potential enzymatic activity of AtXRN4 5′ end 1 and 2 truncated proteins was examined by constitutive expression in a S. cerevisiae xrn1Δ strain engineered to produce PGK1 (FIG. 1D top panel) and MFA2 transcripts (FIG. 1D bottom panel), each bearing a poly(G) tract in the 3′ UTR. The structure of the poly(G) transcripts, and stabilized intermediates that accumulate as a result of Xrn exoribonuclease activity, are shown on the right.

FIG. 2 shows that degradation of transcripts encoding an F-box protein (AtFBL6; At2g25490) is impaired in xrn4 mutants.

FIG. 2A shows that a short 600-700 bp RNA species corresponding to the 3′ end of the AtFBL6 transcript accumulates in AtXRN4 T-DNA insertion mutants (xrn4-5 and 4-4). Full-length transcripts are indicated by FL while short 3′ end transcripts are indicated by 3′. The xrn4-5 mutation is in the Columbia (Col-O) accession and the xrn4-4 mutation is in the Wassilewskija (WS) accession.

FIG. 2B shows a Northern blot analysis of a time course experiment for half-life determination of AtFBL6 mRNAs. Monitoring disappearance of 3′ in both wild-type (Col-O; left panel) and xrn4 mutant (xrn4-5; right panel) indicates that this transcript was stabilized in xrn4-5, and is consistent with a direct role of AtXRN4 in the decay of AtFBL6 (similar results were obtained with the xrn4-4 allele). This experiment shows the highest level of the 3′ end transcript observed in the wild-type but is otherwise representative of three replicates. Since its level are barely detectable, half-life estimation could not be determined for the 3′ shorter transcript in Col-O (ND).

FIG. 2C shows complementation of xrn4 mutants with the AtARN4 gene. T-DNA insertion mutants (xrn4-5 and xrn4-4) were transformed either with an AtXRN4 genomic clone in a pCambia2301 vector (AtXRN4) or with the vector alone (vector). Several independent plant lines from each transformation were analyzed and four representatives are shown. Plants transformed with an AtXRN4 genomic clone showed a decrease in accumulation of 3′ similar to the level observed in wild-type plants (top panels). Presence of full-length AtXRN4 transcripts (bottom panels) in transformed plants was also confirmed using an AtXRN4 cDNA probe. In each case, steady-state level of eIF-4A was used as a control for equal loading.

FIG. 3 shows messenger RNA stability of potential substrates of AtXRN4 in wild-type and mutant seedlings. Total RNA was extracted from Col-0 (left panel) and xrn4-5 (right panel) at various intervals following transcriptional inhibition. Half-life quantification was as in FIG. 2. Expression of expressed protein 172F17 (At4g32020) was detected with EST172F17, bromodomain-like protein (bromo protein; At5g10550) with EST 117J17, ribosomal protein L19p (At5g11750) with EST249B1, and RAP2.4 (Atlg78080) with EST172J24.

FIG. 4 shows that the 3′ end of SCARECROW-LIKE RNA (locus At2g45160) is stabilized in xrn4 mutants.

FIG. 4A shows analysis of SCARECROW-LIKE mRNA stability was analyzed as described herein. A probe complementary to the 3′ end of the SCARECROW-LIKE gene (EST M43D3) was used to detect both full-length transcripts (FL) and 3′ end intermediate RNAs (3′) in blots representative of three experiments. Decay rates of FL in both mutant xrn4-5 (right panel) and Col-O (left panel) were similar, whereas the 3′ transcript was stabilized in xrn4-5. Similar results were obtained for transcripts from other SCARECROW-LIKE genes (data not shown).

FIG. 4B shows that deletion of AtXRN4 does not affect mRNA accumulation. RNA blot analysis of miR171 from A. thaliana wild-type (Col-O and WS) and mutant plants (xrn4-5 and xrn4-4). Ethidium bromide staining of 5S and tRNA is shown below.

FIG. 4C shows mapping of cleavage sites of At2g45160 (SCARECROW-LIKE) and At2g25490 (AtFBL6) by 5′ RLM-RACE. miR171 and SCARECROW-LIKE transcript share perfect complementarity. No known mRNA or other 21-25 nt small RNA whose predicted target transcript would include AtFBL6 has been detected or has been reported so far. The number of RACE clones sequenced corresponding to each cleavage site is indicated above vertical arrowheads.

FIG. 5 shows expression analysis of predicted mRNA target transcripts in inflorescence tissues of wild-type and the xrn-4-5 mutant. Poly(A)⁺ RNA was column-purified and 1 μg was fractionated on 1.1% agarose gel before transfer to nylon membrane.

FIG. 5A shows that mRNA-mediated cleavage products of ARF10 (At2g28350), ARF17 (At1g77850), MYB33 (At5g06100), MYB6S (At3g11440), and PHV (At1g30490) are accumulating in xrn4-5 mutant.

FIG. 5B shows that steady-state levels of ARF8 (At5g37020), AP2-like (At4g36920), TCP2 (At4g18390) and TCP4 (At3g15030), SPL10 (At1g27370), and AGO (At1g48410) were unchanged in Col-O and xrn4-5. eIF4A was used as a reference for equal loading. Asterisks indicate products generated by mRNA cleavage that are elevated in the mutant. ³²P-labeled probes were PCR amplified using primers upstream and downstream of the cleavage site allowing detection of both full-length transcripts and putative cleavage products.

FIG. 6 shows a model depicting the function of 5′ to 3′ exoribonuclease AtXRN4 in degrading the 3′ products produced by mRNA-directed cleavage. The 3′ end of selected transcripts is degraded in a 5′ to 3′ direction by AtXRN4 (or functionally homologous XRNs in other systems), unless the corresponding XRN gene is inactivated. Evidence also suggests that an alternative decay pathway is present.

### DETAILED DESCRIPTION OF THE INVENTION

Applicants have discovered that inactivation of the gene for a cytoplasmic 5′ to 3′ exoribonuclease results in the accumulation of diagnostic RNA cleavage products (intermediates) corresponding to the transcripts that are targeted for cleavage by complementary mRNA. Treatment of the mutant with a chemical that inhibits RNA synthesis can enhance the accumulation of the intermediate relative to that of controls.

RNAi approaches involving siRNA-directed mRNA degradation have been an effective means to study gene function in a variety of multicellular eukaryotes, and numerous natural mRNAs have also been identified. siRNAs and mRNAs are thought to have a broad range of regulatory functions, including triggering degradation of unknown target mRNAs With the identification of many mRNA and siRNAs by cloning approaches in various systems, a current challenge is to identify the modes of action of these RNAs and their corresponding targets. xrn mutants can contribute to achieving this goal by enhancing the accumulation of 3′ end intermediates for transcripts targeted by mRNA- or siRNA-mediated cleavage. This approach would be complementary to, or in many cases advantageous to current methods such as cloning mRNAs, computational prediction of target sequences, and cloning of 5′ RACE products.

At least in higher plants, imperfect sequence complementarity between mRNA and its target mRNA can also trigger cleavage of full-length transcripts, making computational predictions difficult. The accumulation of intermediates in an xrn4 mutant would identify mRNA targets while providing direct evidence for a cleavage mechanism, and be adaptable to large-scale genomic approaches such as DNA microarray analysis.

The degradation of mRNA is an essential step in gene expression that, in multicellular organisms, can be regulated by siRNAs or mRNAs. These small RNAs guide cleavage of complementary mRNAs by the RISC complex or a closely related miRNP. However, the yeast system (Saccharomyces cerevisiae), which has been the source of most of our knowledge of in vivo eukaryotic mRNA degradation mechanisms, lacks mRNAs and RNAi capability. Using reverse genetics in combination with microarray analyses, we have identified multiple substrates of AtXRN4, the Arabidopsis homolog of the major yeast mRNA degrading exoribonuclease, Xrn p. Insertional mutation of AtXRN4 leads to accumulation of RNA species corresponding to the 3′ region of several mRNAs including the AtFBL6 transcript. This accumulation correlates with increased stability of the 3′ end of the transcript and is reversed following complementation with the wild-type AtXRN4 gene. Furthermore, we present in vivo evidence that xrn4 mutants accumulate similar 3′ short RNA fragments corresponding to decay intermediates of mRNA-mediated cleavage of SCARECROW-LIKE transcripts in seedlings and several other mRNA target transcripts encoding transcription factors in inflorescence tissues. This provides strong evidence that AtXRN4 degrades mRNAs, including the 3′ end of some transcripts produced by mRNA-directed cleavage, and that homologous enzymes may serve a similar function in other multicellular eukaryotes.

The mutants useful in the invention are eukaryotic cells or non-human organisms in which the gene encoding the enzyme AtXRN4 or its homologue is non-functional, such that AtXRN4 or its homologue are not produced or are non-functional. Suitable types of eukaryotic cells include plant, bacterial, and mammalian cells. Suitable non-human organisms include plants and mammals. The invention has been exemplified using Arabidopsis thaliana mutants that contain a T-DNA insert in the gene encoding the enzyme XRN4 that disrupts expression of the gene and renders it non-functional. A. thaliana mutants useful in the invention include mutant xrn4-5 obtained from the Syngenta Arabidopsis Insertion Library (SAIL) (Syngenta Biotechnology, Inc., Research Triangle Park, North Carolina) and xrn4-4 obtained from the University of Wisconsin T-DNA-tagged lines (Knock-out Facility at the University of Wisconsin, Madison, Wis.). (GARLIC_{—}681_E01.b.1a.Lb3Fa).

Other mutants can be created by interrupting the AtXRN4 gene with non-coding sequences, such as T-DNA sequences, removing the AtXRN4 gene or replacing it with a non-functional sequence that has been truncated or otherwise altered.

As used herein “defective AtXRN4 gene”, “defective homologue of AtXRN4” and similar terms refer to genes encoding XRN4 or its homologues that are defective, such that XRN4 or its homologue is not produced, or is produced in whole or in part but is non-functional or partially functional.

Homologue of AtXRN4 refers to a protein having the same or similar 5′ to 3′ exoribonuclease enzymatic activity as Arabidopsis thaliana XRN4 (a functional homologue) and/or related DNA sequence (a genetic homologue). Generally, a homologue is from a species other than Arabidopsis thaliana, but other A. thaliana proteins having the same or similar 5′ to 3′ exoribonuclease enzymatic activity or similar sequence as A. thaliana XRN4 are also included in as homologues. Homologues include Xrn1p. XRN homologs are present in a number of other organisms, including plants and mammals.

The invention provides methods of identifying aberrant RNA or RNA targeted for cleavage by mRNA or siRNA comprising the steps of optionally treating mutant cells or a non-human organism in which the gene encoding AtXRN4 or its homologue is defective with an agent that inhibits RNA synthesis; isolating RNA from the cells or non-human organism; and identifying aberrant RNA or RNA cleavage products in the isolated RNA by comparison with the wild type cells or non-human organism.

In the practice of the methods of the invention, one or more aberrant RNA species or RNA cleavage products can be identified in the RNA isolated from mutant cells or non-human organisms. Reference to identifying the isolated RNA as aberrant RNA or an RNA cleavage product thus includes the situation wherein only one transcript in the isolated RNA is identified as well as the situation where two or more transcripts in the isolated RNA are identified.

In the performance of the methods of the invention, the mutant eukaryotic cells or non-human organism are optionally treated with an agent that inhibits RNA synthesis such as cordycepin. Treatment of the mutant cells or non-human organism with a chemical that inhibits RNA synthesis can enhance the accumulation of the intermediate relative to that of controls. However, inhibiting RNA synthesis prior to purifying RNA from the mutant cells or organism is not required.

After optional treatment of the mutant cells or non-human organism with an agent that inhibits RNA synthesis, RNA is purified from the mutant cells or non-human organism; RNA can be purified from the cells or tissue from the non-human organism using standard techniques known in the art.

Aberrant RNA or RNA cleavage products in the isolated RNA are then identified. Generally, the isolated RNA is compared with RNA from wild type cells or non-human organism, and differences in the abundance of transcripts and/or the presence or absence of transcripts between the mutant and wild type are determined. Transcripts of interest in the isolated RNA from mutant cells or non-human organism are then characterized as aberrant RNA or RNA cleavage products by size and by comparison with known nucleic acid sequences such as genes encoding mRNA encoding, cDNA sequences, aberrant RNA sequences, mRNA sequences or an siRNA sequences.

Wild type cells or non-human organism refers to cells or non-human organisms of the same or similar type as the mutant cells or non-human organism in which the AtXRN4 gene or its homologue is not defective.

Aberrant RNA refers to RNAs that differ in size or polarity from the capped and polyadeylated RNA produced in wild type.

Identification of transcripts in the isolated RNA can be done by techniques such as microarray analysis, RT-PCR followed by sequencing of the DNA corresponding to the intermediates and comparison with known sequences, Northern blot analysis with agarose or polyacrylamide gels, or by hybridization to probes indicative of a known gene.

In one preferred embodiment of the methods of the invention, the identifying step comprises the steps of preparing microarray probes from the isolated RNA; hybridizing the microarray probes with a microarray comprising DNA that represents gene transcripts from the same or similar type of cells or non-human organism; detecting changes in hybridization of the microarray probes from the mutant cells or non-human organism relative to that of the wild-type; relating changes in the hybridization signal of a probe with the identity of the corresponding genes represented on the microarray; and characterizing the hybridized RNA as aberrant RNA or an RNA cleavage product by its size on Northern Blots and/or by comparison with known nucleic acid sequences such as genes encoding mRNA, encoding, cDNA sequences, aberrant RNA sequences, mRNA sequences or siRNA sequences.

In another preferred embodiment of the invention, the identifying step comprises the steps of preparing probes corresponding to at least one gene or other nucleic acid sequence of interest; hybridizing the probes with a Northern blot that contains isolated RNA from the mutant cells or non-human organism and wild type RNA from the same or similar type of cells or non-human organism; detecting hybridization of the probes with RNA on the Northern blot; and characterizing the isolated RNA as aberrant RNA or an RNA cleavage product by size and comparison with known nucleic acid sequences, such as genes encoding mRNAs, cDNA sequences, aberrant RNA sequences, mRNA sequences or siRNA sequences.

The characterizing step preferably comprises the use of Northern blot, Rapid Amplification of Complementary Ends (RACE), reverse transcriptase-polymerase chain reaction (RT-PCR) or DNA sequencing and/or oligo-directed RNaseH cleavage in characterizing the isolated RNA as aberrant RNA or an RNA cleavage product.

The methods of the invention result in the rapid accumulation of RNA resulting from mRNA cleavage to levels that can be detected by standard techniques for detecting nucleic acids and the identity of transcripts determined.

The methods of the invention can be used to identify aberrant RNA and/or RNA cleavage products that are related to diseases or other medical or pathological conditions. The methods of the invention are thus also useful for diagnosis of such conditions.

### EXPERIMENTAL

Arabidopsis thaliana T-DNA Insertion Lines

Two separate approaches were taken to identify Arabidopsis lines containing T-DNA inserts in the AtXRN4 gene. First, a reverse genetic approach using PCR-based screening of the second collection of the Arabidopsis Knock-out Facility at the University of Wisconsin was pursued (Krysan et al., 1999). Forward PG820 (5′-ATACCCGAAGTCAATTAGTGACGTCGTTTG-3′) and reverse PG821 (5′-TGGACTACTGTTCATGACGAATTCCTTTG-3′) primers were used for screening of the Wisconsin insertion collection following standard protocols (Krysan et al., 1999). Second, taking advantage of a large collection of sequenced T-DNA insertions available to the research community from the Syngenta Arabidopsis Insertion Library (SAIL), the database was searched for T-DNA insertions in the AtXRN4 gene using a BLAST analysis (Sessions et al., 2002). Once the seeds corresponding to potential AtXRN4-disrupted lines were obtained and grown, individual plants were screened by PCR for homozygous xrn4 mutants using PG820 and PG821 primers, and confirmed by Southern Blot. From these screens, xrn4-4 was obtained from the University of Wisconsin T-DNA-tagged lines while xrn4-5 was obtained from SAIL.

Microarray Analysis

The 15K cDNA microarray slides were generated at the Genomics Technology Support Facility (GTSF) at Michigan State University. The 15,532 ESTs were spotted on supper-manine glass slides (Telechem International Inc., Sunnyvale, Calif.). Before use, the slides were re-hydrated, UV-crosslinked at 90 mJ and blocked as recommended by the manufacturer. Thirty micrograms of total RNA, extracted from two week-old Col-O and xrn4-5 seedlings treated with cordycepin for 120 minutes, were aminoallyl labeled with Cy3 or Cy5 fluorescent dye according to The Institute for Genomic Research protocol (http://www.tigr.org). Cy3 and Cy5 probes were then resuspended in 4 μl of 10 mM EDTA, mixed with 50 μl of SlideHyb buffer 2 (Ambion, Austin, Tex.), and loaded onto the slide. The slides were hybridized overnight in a 55° C. water bath. Once washed and dried, the slides were scanned with a GenePix 4000B scanner and fluorescent signals analyzed using GenePix Pro 4.1 software (Axon Instruments, Union City, Calif.). Once the quality of the hybridization was evaluated and the raw data extracted, the intensity values were normalized using the Global Lowess Method. Intensity ratios, defined as the signal intensity in xrn4-5 versus Col-O, were determined. Then, the number of clones with a ratio>1.5 in three out of the four slides was identified. A total of four slides corresponding to two biological experiments were used for the experiment. Each microarray hybridization included two slides, the experimental and its technical repetition or reverse-labeling.

Half-life Measurements, Nucleic Acid Isolation and Blot Analysis

Cordycepin time course experiments were carried out to determine half-lives as described (Gutierrez et al., 2002). Tissue samples were harvested at regular intervals following cordycepin treatment, and total RNA was extracted using Trizol reagent according to the manufacturer's instructions (Invitrogen, Carlsbad, Calif.). Blot hybridization of total RNA was performed as previously described (Newman et al., 1993). Poly(A)⁺ RNA was column-purified from total RNA extracted from inflorescence tissue using Oligotex mRNA kit (Qiagen, Valencia, Calif.) according to the manufacturer's instructions.

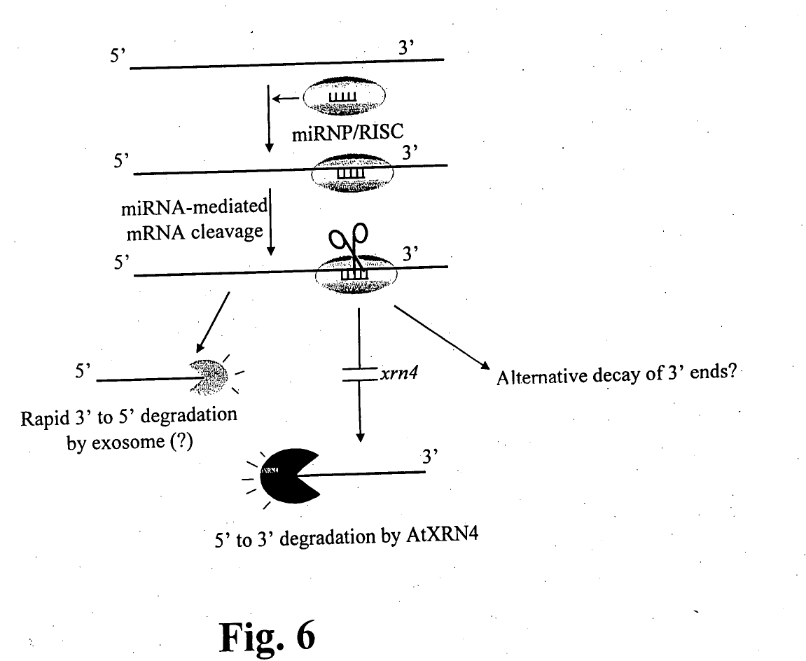

Radiolabeled probes corresponding to AtFBL6 were prepared using Arabidopsis EST clones from the PRL2 EST collection (Newman et al., 1994). EST 171N2T7 (0.9 kb) was used as the 3′ probe while the 5′ probe (1.1 kb) was amplified using EST 118L22T7 as a template and T7 and PG1628 (5′-GCCCTTTCCAACAGATTCAA-3′) as primers. All the other probes used to monitor transcript stability were amplified from ESTs mentioned in the figure legends using T7 and SP6 as primers. All ESTs were obtained from the Arabidopsis Biological Resource Center (ABRC). Radiolabeled probes used to examine the expression of mRNA predicted target genes were amplified by PCR using gene specific primers and covered the 3′ proximal region of the gene (see FIG. 6 legend). The primer list is available upon request.

For the analysis of mRNAs, fifty μg of total RNA was separated on a denaturing 15% polyacrylamide gel containing 8M urea, electrophoretically transferred to a Hybond-N+ membrane (Amersham, Piscataway, N.J.), and then hybridized as described (Llave et al., 2002b; Park et al., 2002). ³²P-5′-end-labeled oligonucleotide complementary to miR171 (5′-GATATTGGCGCGGCTCAATCA-3′) was used as a probe.

Mapping of the mRNA Target Cleavage Sites by 5′ RACE

Cleavage site sequences were determined using the FirstChoice® RLM-RACE kit (Ambion, Austin, Tex.) without prior phosphatase treatment. cDNA synthesis was performed using total RNA from xrn4-5 seedlings primed with oligo(dT). Gene-specific PCR amplification was carried out with the 5′ RACE Outer Primer (supplied by the manufacturer) and 3′ primers specifically design to amplify either At2g45160 (PG 1947-^{5′}AGGCGACGGAGTTTACTGGAAG^{3′}) or AtFBL6 (PG 1687-^{5′}CAAAAGCCAGAGCAAACCTCTGA^{3′}). The 5′ RACE products were purified and ligated. Multiple independent clones were then sequenced, and mapping of cleavage sites confirmed or established.

Heterologous Expression and Exoribonuclease Activity in Yeast

Full-length AtXRN4 and 5′ ends 1 and 2 were constitutively expressed using a high copy plasmid in an xrn1 mutant yRP884 (MATα, trp1-Δ1, ura3-52, leu2-3, 112, lys2-201, cup::LEU2 pm, XRN1::URA3), generously provided by Dr. Roy Parker (Department of Molecular and Cellular Biology, University of Arizona, Tucson, Ariz.). Wildtype yRP841 (MATα, trp1-Δ1 ura3-52 leu2-3, 112 lys2-201 cup::LEU2 pm) served as a control. Analysis of poly(G) reporter RNAs was performed as described (Kastenmayer and Green, 2000; Kastenmayer et al., 2001).

Plasmid Construct and Transformation of Arabidopsis

To obtain a full-length genomic AtXRN4 clone, an A. thaliana λ ZAP®II genomic library (Stratagene, La Jolla, Calif.) was screened according to the manufacturer's instructions. Two separate plasmids, containing different portions of the AtXRN4 gene, were excised from the library: the larger insert encompassing a 5.4 kb region from exon 1 to exon 20, including part of intron 20 as well as a 1.8 kb region upstream of the ATG start codon, and the smaller one including the last two exons as well as a 2 kb region downstream of the stop codon. The two inserts were ligated together to create a full-length genomic AtXRN4 clone that was subcloned into pCambia 2301. The insert of the full-length clone was sequenced to confirm proper orientation and integrity. Transformation with Agrobacterium tumefasciens C58 was performed as previously described (Bariola et al., 1999).

Results

Identification of T-DNA Insertion Lines to Assess the Role of AtXRN4 in Degrading mRNAs

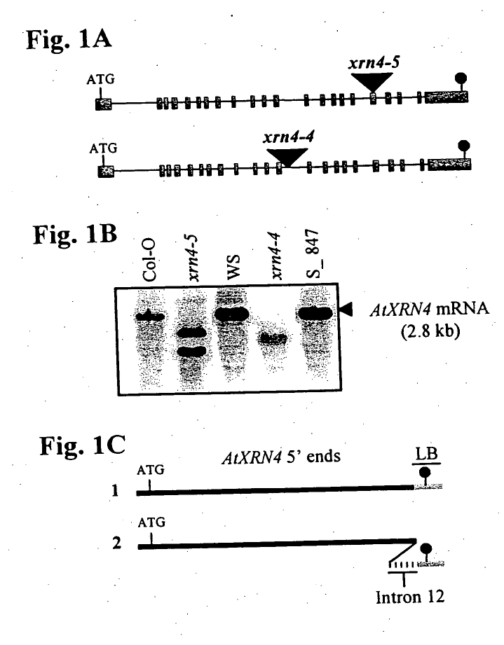

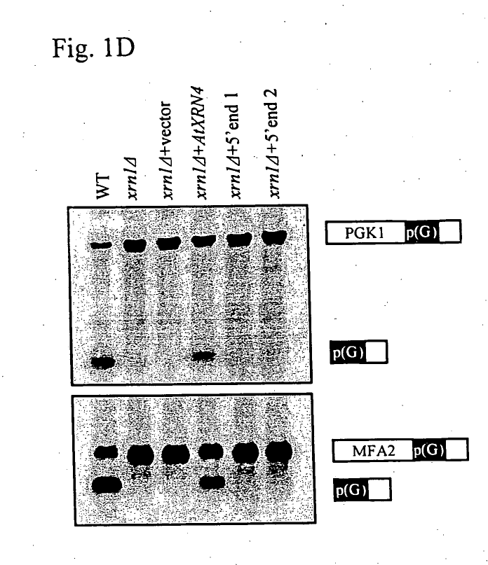

As a first step to identify substrates of XRNs in multicellular organisms, a reverse genetic approach in Arabidopsis was taken. Since AtXRN4 is the best candidate for a functional homolog of Xrn1p (Kastenmayer and Green, 2000; Kastenmayer et al., 2001), we sought to identify T-DNA inserts in the AtXRN4 gene via database searches and PCR screening of available T-DNA insertion collections (Krysan et al., 1999; Sessions et al., 2002). Among several alleles identified, two homozygous T-DNA insertion lines, xrn4-4 and xrn4-5, were obtained, confirmed by southern blotting (data not shown), and further characterized (FIG. 1A). The T-DNAs in these mutants disrupt introns or exons in the coding region, and neither mutant had any obvious phenotype.

To test whether xrn4-4 and xrn4-5 mutations are null mutants, we examined whether they disrupt expression of AtXRN4. Expression of full-length AtXRN4 mRNA is observed in wild-type Arabidopsis plants (Col-O and WS) as well as in a SAIL line containing a T-DNA insertion elsewhere in the genome (S_{—}847), but is undetectable in homozygous AtXRN4-disrupted lines (xrn4-5 and 4-4) (FIG. 1B). As expected for T-DNA insertions downstream of the promoter, truncated AtXRN4 transcripts were observed in the mutants (FIG. 11B). Therefore, we investigated whether these truncated RNAs could make functional proteins in vivo. Two cDNAs, AtXRN4 5′ end 1 and 2, corresponding to truncated transcripts containing the 5′ end of the AtXRN4 transcript fused to sequences derived from the T-DNA, were cloned from xrn4-4 plants (FIG. 1C). These cDNAs were produced in yeast cells engineered to express mRNAs containing poly(G) tracts. In yeast, poly(G) tracts block the progression of XRN enzymes resulting in a mRNA decay intermediate in wild-type cells, or in xrn1Δ cells when full-length AtXRN4 is expressed (FIG. 1C; (Kastenmayer and Green, 2000; Kastenmayer et al., 2001). However, similar to what was observed in xrn1Δ transformed with vector alone, constitutive expression of AtXRN4 5′ end 1 or 2 truncated proteins failed to produce detectable exoribonuclease activity and therefore did not complement the xrn1Δ mutant (FIG. 1C). Consequently, the truncated AtXRN4 products are unlikely to be active. This is not surprising since similar mutations in the XRN1 gene produce a non-functional protein (Page et al., 1998). These results and those of the complementation experiments below provide strong evidence that both xrn4-4 and xrn4-5 are loss of function mutants.

Insertional Mutation of AtXRN4 Does Not Significantly Affect the Degradation of Randomly Selected Unstable Transcripts

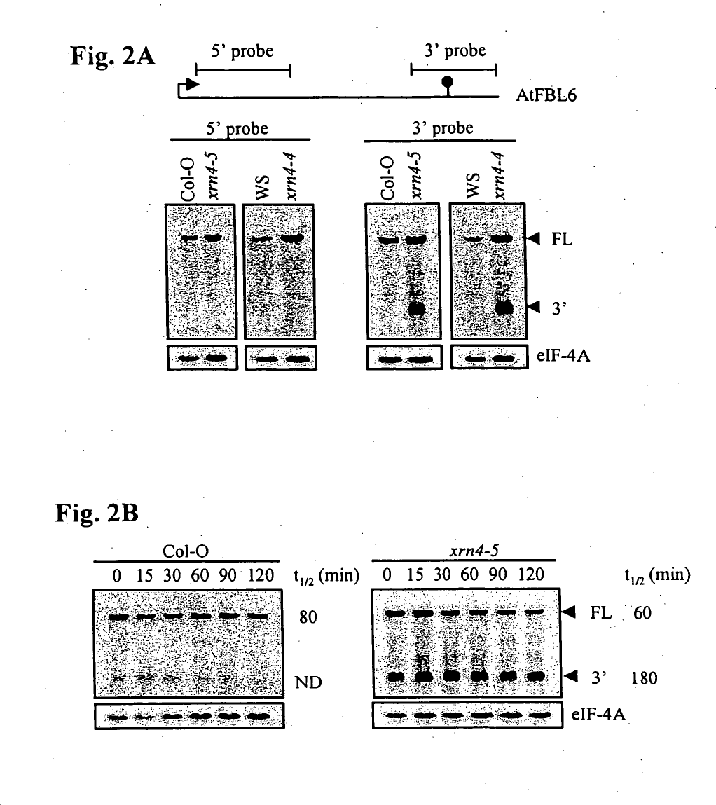

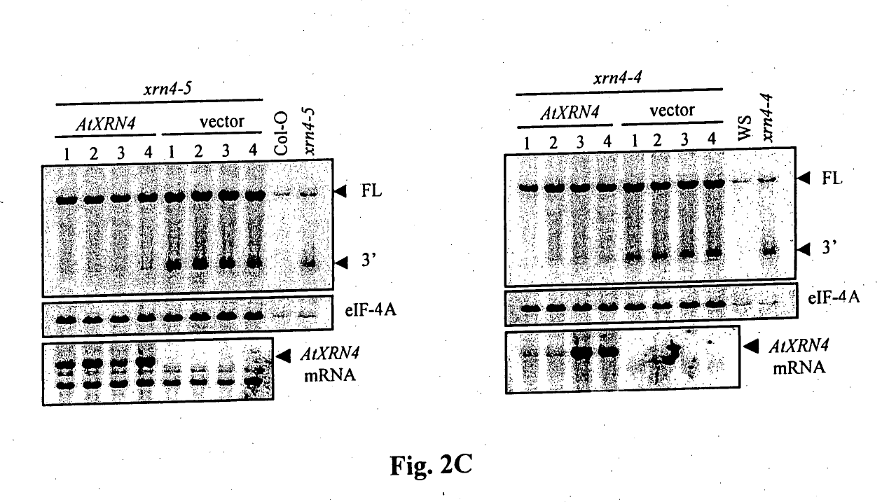

To begin assessing the role of AtXRN4 in cytoplasmic transcript degradation and its effect on global RNA accumulation, several genes known to encode unstable transcripts (AtGUTs), with half-lives of less than 60 minutes, were randomly chosen from those identified by Gutierrez et al. (2002). AtGUTs are thought to be involved in various physiological and developmental processes that may benefit from the rapid control afforded by unstable transcripts. Therefore, we first investigated whether the cytoplasmic 5′ to 3′ exoribonuclease, AtXRN4, was involved in the general turnover of AtGUTs. To this end, the stability of five transcripts known to be unstable including a nitrate reductase (At1g37130), an expansin family member (At3g45970), a senescence associated gene (At4g35770), a zinc finger family member (At3g54810), and an expressed protein (At2g40000) were monitored in the wild-type and the xrn4 mutant following transcriptional inhibition (FIG. 2). Compared to their half-lives in the wild-type (Col-O), the five AtGUTs examined in this study showed similar decay rates in the mutant plants (xrn4-5). This indicates that either AtXRN4 does not play a significant role in controlling the degradation of these unstable transcripts in A. thaliana or that an alternative decay pathway is present to prevent the accumulation of these transcripts when AtXRN4 is not functional.

DNA Microarray Analysis First Indicates that AtFBL6 Transcript is a Potential Substrate of AtXRN4

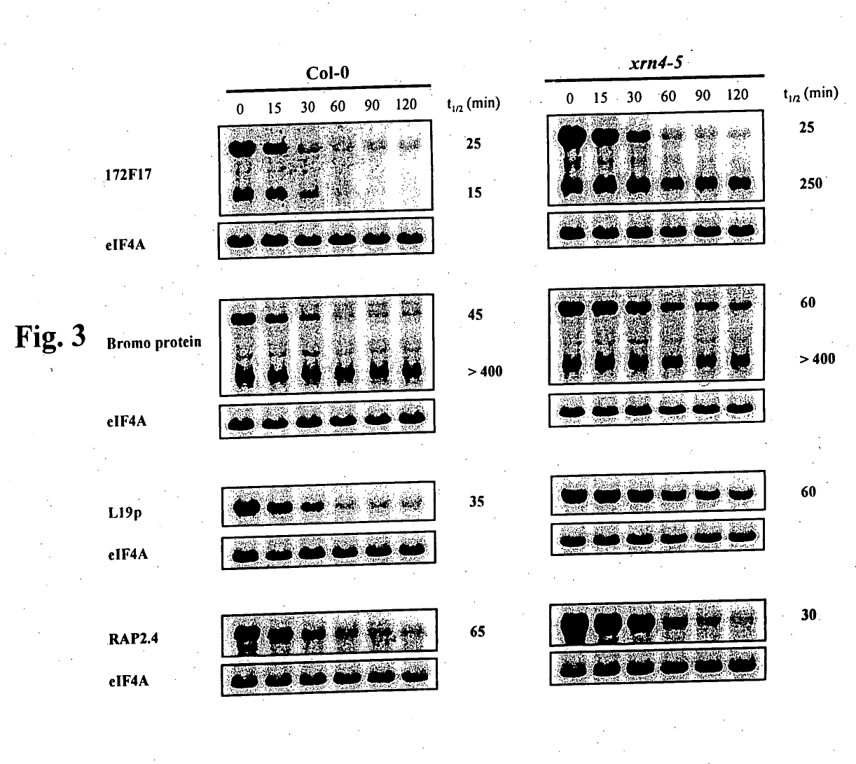

To identify substrates of AtXRN4 and evaluate its functional role, cDNA microarrays containing about 15K ESTs were used to compare xrn4-5 and the wild type (Gutierrez et al., 2002; Perez-Amador et al., 2001). Samples were harvested 120 minutes after transcriptional inhibition to help visualize mRNA decay differences. Two biological replicates, each with reverse-labeling technical replicates were performed for a total of 4 slides. Following data analysis, mRNAs corresponding to 19 ESTs (14 genes) were found to accumulate mRNA levels greater than 1.5-fold in the mutant compared to the wild-type in at least 3 out of the 4 slides (Table 1). Most of them had unclassified biological function, based on the Munich Information Center for Protein Sequences (MIPS). Interestingly, two different ESTs on the 15K cDNA microarray (171N2T7 and 96O1T7) showed increased abundance (between 2.3 and 3.3-fold) in the mutant relative to the wild-type in all four slides, and an additional EST (203J17T7) was found to be elevated in three out of the four slides. All three ESTs correspond to a single transcript that encodes an F-box protein containing leucine-rich repeats (AtFBL6; At2g25490) that could represent a possible substrate for AtXRN4. To confirm the microarray results, AtFBL6 transcript accumulation in the wild-type and xrn4 plants was monitored using RNA blot analysis (FIG. 3A). While the AtFBL6 transcript was increased in abundance in the mutant as expected, more interesting was the striking appearance of an abundant low molecular weight RNA species in the mutant (FIG. 2A). A probe spanning the 5′ end of AtFBL6 (5′ probe) only hybridizes to the full-length transcripts while a probe complementary to the 3′ end (3′ probe) detects both the full-length and the shorter transcript in both wild-type and mutant plants. This is consistent with the smaller RNA corresponding to a decay fragment from the 3′ end of AtFBL6 (FIG. 2A).

TABLE 1
Potential targets of AtXRN4 as identified by cDNA microarrays.

| ID | Locus | Gene Name | MIPS Function |
|---|---|---|---|
| 171N2T7 | At2g25490 | AtFBL6 | Unclassified |
| 96O11T7 | At2g25490 | AtFBL6 | Unclassified |
| 203J17T7 | At2g25490 | AtFBL6 | Unclassified |
| 172J24T7 | At1g78080 | RAP2.4 | Transcription factor |
| 113E4T7 | At1g78080 | RAP2.4 | Transcription factor |
| 172F17T7 | At4g32020 | Expressed | Unclassified |
| 91A8T7 | At4g32020 | Expressed | Unclassified |
| 246B4T7 | At4g32020 | Expressed | Unclassified |
| 94I2T7 | At5g16110 | Expressed | Unclassified |
| 220J17T7 | At3g17510 | CBL-interacting protein kinase I | Signal transduction |
| 285B1T7 | At2g25250 | Expressed | Unclassified |
| 128D24XP | At3g05220 | heavy-metal-associated domain- | Unclassified |
| | | containing protein | |
| 231A14T7 | At4g00780 | MATH domain-containing protein | Unclassified |
| G9F10T7 | At1g51680 | 4-coumarate: CoA ligase I | Secondary metabolism |
| 249B1T7 | At5g11750 | Ribosomal protein L19p | Ribosome biogenesis |
| 117J17T7 | At5g10550 | Bromodomain-like protein | Transcriptional control |
| 190N23T7 | At5g44190 | Myb family transcription factor | Signal transduction |
| 164E15XP | At1g73490 | RNA recognition motif containing | Unclassified |
| | | protein | |
| 78H6T7 | At1g73600 | Phosphoethanolamine N- | Lipid, fatty-acid and |
| | | methyltransferase related protein | isoprenoid metabolism |

As the 3′ end of AtFBL6 can also be detected at very low level in wild-type plants (e.g. FIG. 2B), we examined the possibility that its prominence in xrn4 plants was due to stabilization in the mutants. Changes in abundance of AtFBL6 transcripts in seedlings were monitored during a 120 min time course after transcriptional inhibition and decay kinetics analyzed (FIG. 2B). The full-length transcripts were moderately stable and their half-lives differed by less than two fold in the wild-type and the mutant plants with t_{/2}=80 min and 60 min, respectively. In contrast, the AtFBL6 3′ end transcript appears notably more stable in the xrn4 seedlings (t_{1/2}=180 min) than in wild-type in multiple experiments, although its low level in the wild-type precludes an accurate determination of its decay rate (ND; see FIG. 2 description). These results, taken together with the in vivo 5′ to 3′ exoribonuclease activity displayed by AtXRN4 (Kastenmayer and Green, 2000), indicate that the 3′ end RNA fragment of AtFBL6 is a likely substrate of AtXRN4.

To confirm that the increase in accumulation of the 3′ end of AtFBL6 was caused by the insertional mutation in the AtXRN4 gene, complementation experiments were performed to determine if this would reverse the molecular phenotype. When plants homozygous for either the xrn4-5 or the xrn4-4 allele were transformed with an AtXRN4 genomic clone, all four transformants examined for each mutant expressed AtXRN4 mRNA in contrast to the vector controls (FIG. 2C, bottom panels). Moreover, in the AtXRN4 transgenic plants, accumulation of the 3′ end short AtFBL6 transcript was markedly reduced to levels similar to those observed in wild-type plants (FIG. 2C, top panels). This showed that the increase in accumulation of this RNA species was a result of the mutations of AtXRN4. The stabilization of the 3′ fragment of AtFBL6 mRNA in the AtXRN4-disrupted lines provided initial evidence that XRN enzymes, previously known to degrade mRNAs only in yeast, are also involved in the degradation of mRNAs in Arabidopsis.

Potential AtXRN4 Substrates Exhibit Several mRNA Decay Patterns

A relatively low cut-off ratio of 1.5 was chosen to identify transcripts with elevated abundance in the mutant compared to the wild-type in the microarray experiments. Therefore, Northern blot analyses were also carried out to validate potential targets found with microarrays, and specifically identify those transcripts affected at the mRNA stability level (Table 1). The accumulation of four transcripts that increased in the xrn4-5 mutant was analyzed at different intervals following transcription inhibitor treatment and half-lives determined both in the wild-type (Col-0) and in the mutant (xrn4-5). Overall, the relative level of expression, examined two hours after transcription inhibition, was in agreement with the results obtained from the microarray analysis (FIGS. 2 and 3, at t=120 min).

The mRNA decay kinetics were then analyzed to directly assess the effect of AtXRN4 inactivation on mRNA stability. Interestingly, and reminiscent of our previous observations with AtFBL6 transcript, was the presence of a long transcript and a short RNA fragment detected with probes for both expressed protein 172F17 and a bromodomain-like protein (FIG. 3). Although some naturally-occurring decay intermediates in plants have been observed in few cases (Higgs and Colbert, 1994; Tanzer and Meagher, 1995), decay intermediates are rarely detectable in higher eukaryotes and can not even be engineered to accumulate with insertion of a poly(G) tract as done in yeast (Kastenmayer and Green, 2000; Kastenmayer et al., 2001). Moreover, similar to AtFBL6 transcript, the lack of AtXRN4 had little effect on the stability of the full-length transcript encoding expressed protein 172F17, yet the half-life of the smaller transcript was markedly increased in the xrn4-5 seedlings (t_{1/2}=250 min compared to 15 min in Col-O). This indicates that the shorter transcript of 172F17 like that of AtFBL6, is likely a substrate of AtXRN4 (FIGS. 2 and 3). A third potential AtXRN4 substrate, the mRNA for bromodomain-like protein, also accumulated a short RNA transcript. However, in this case, the short RNA was too stable in the wild-type (t_{1/2}>400 min) to determine if it became more stable in the xrn4-5 mutant (FIG. 3). Short RNA fragments were not observed for two other transcripts identified on the arrays, RAP2.4 and ribosomal protein L19p (FIG. 3). The L19p gene may still be a substrate of AtXRN4 because its full-length transcript was about twice as stable in the xrn4-5 mutant compared to wild-type (t_{1/2}=60 min and t_{1/2}=35 min, respectively). Other transcripts in Table 1 have similar properties (data not shown). For RAP2.4, the major impact of deleting AtXRN4 appears indirect. The mRNA level is elevated at time zero most likely due to increased transcription, and if anything is less stable in the xnr4-5 mutant.

Potential Role of AtXRN4 in Degrading mRNA-Mediated mRNA Cleavage Product

Very few transcripts were affected by AtXRN4 deletion on microarrays, and that three out of four likely substrates of AtXRN4 identified also accumulated 3′ end RNA fragments. In particular, the accumulation of the 3′ end fragment of AtFBL6, 172F17, and bromodomain protein RNAs, and the absence of detectable 5′ end RNA species was reminiscent of a recent report of sequence-specific cleavage directed by mRNAs in Arabidopsis (Llave et al., 2002b). Degradation of SCARECROW-LIKE RNAs proceeds through the mRNA-directed cleavage pathway mediated by miR171, that is characterized by the accumulation of the 3′ end of the RNAs while the corresponding 5′ end is undetectable (Llave et al., 2002a; Llave et al., 2002b). We hypothesized that, since miRNP-mediated cleavage produces 3′ products with 5′ monophosphates (Hannon, 2002; Llave et al., 2002b), these 3′ cleavage products would be potential RNA substrates for a 5′ to 3′ exoribonuclease such as AtXRN4. If so, we would expect these short RNA species to be stabilized in xrn4 mutants.

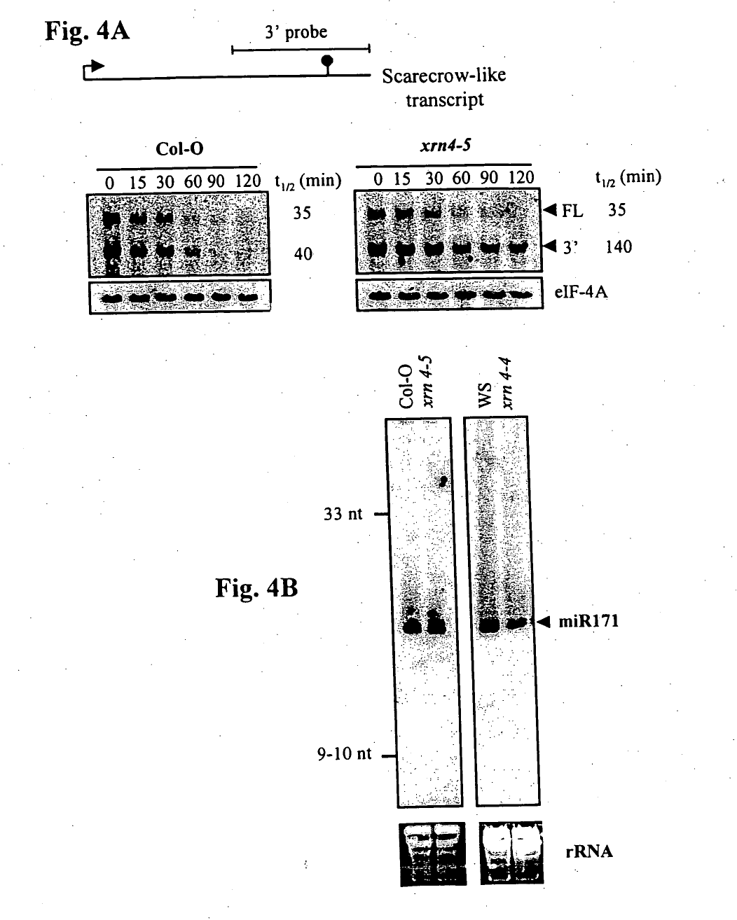

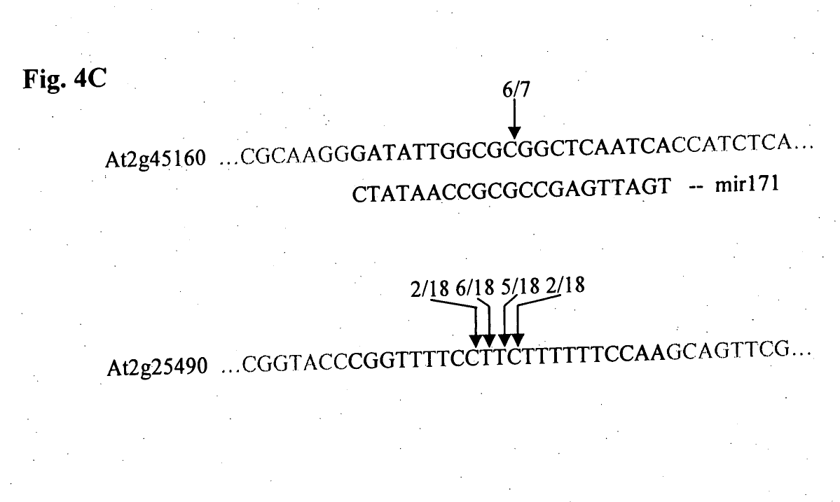

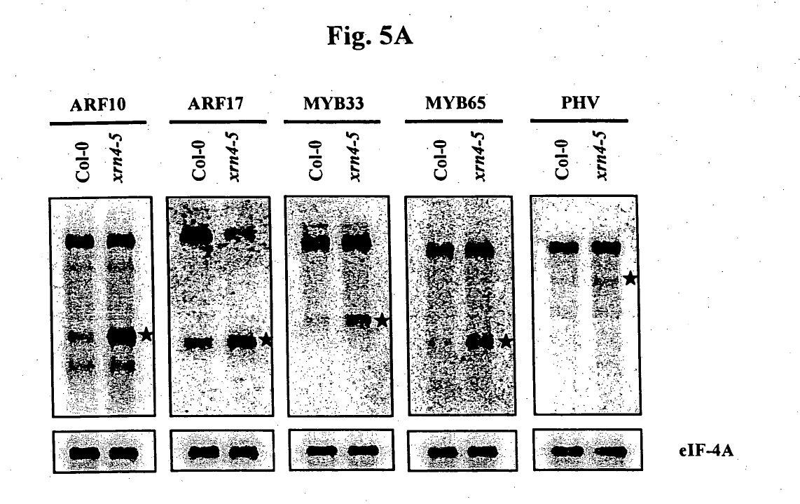

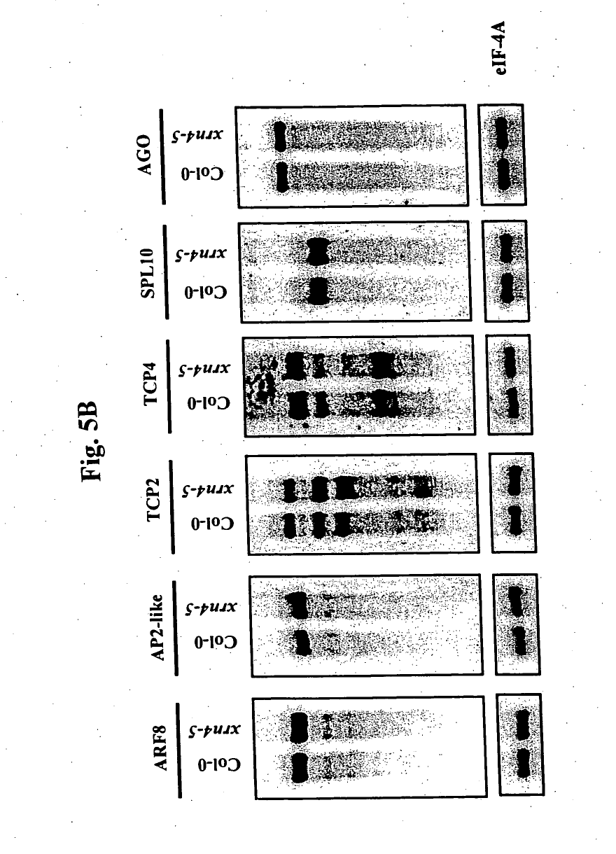

To investigate whether AtXRN4 could potentially be involved in degrading the 3′ RNA products resulting from mRNA-directed mRNA cleavage, we monitored the decay of both full-length and 3′ end short SCARECROW-LIKE RNA species (locus At2g45160) on Northern blots following transcriptional inhibition in wild-type and xrn4-5 plants (FIG. 4A). Messenger RNA half-life measurements indicated that the stability of the full-length SCARECROW-LIKE transcripts was similar in wild-type and in the xrn4-5 insertion line (t_{1/2}=35 min), whereas the apparent stability of the 3′ end RNA was markedly increased in the mutant (t_{1/2}=140 min in xrn4-5 compared to t_{1/2}=40 min in wild-type; FIG. 4A). This indicates that decay of the RNA corresponding to the 3′ end of the SCARECROW-LIKE transcript was noticeably reduced in xrn4 plants. Similar observations were made for other SCARECROW-LIKE family members (loci At3g60630 and At4g00150; data not shown), all proposed specific targets of the 21nt-miR171 (Llave et al., 2002a; Llave et al., 2002b; Reinhart et al., 2002; Rhoades et al., 2002). In contrast to the 3′ end of SCARECROW-LIKE mRNAs, miR171 levels are unaffected in xrn4-5 mutants (FIG. 5B). These results indicate that disruption of AtXRN4 increases the accumulation of the SCARECROW-LIKE 3′ end RNAs by decreasing their rate of decay, without altering miR171 abundance.

To confirm that the 3′ RNAs stabilized in xrn4-5 seedlings were generated by miR171-directed SCARECROW-LIKE mRNA cleavage, we mapped the 5′ ends by an RNA ligase-mediated 5′ RACE method as described by Llave et al. (2002b). We found most of the sequences ended in the middle of the region of pairing between miR171 and its target RNA as expected for predicted sites of mRNA-mediated cleavage (FIG. 4C, top panel; Liave et al., 2002b). These findings validate the shorter RNA fragments detected on Northern blots as the 3′ end transcripts that arose from cleavage of the SCARECROW-LIKE transcript by miR171.

Since several additional mRNA target transcripts have recently been experimentally validated, we further investigated the role of AtXRN4 in degrading mRNA cleavage products mediated by the corresponding mRNAs. Predominant targets of plant mRNA include several transcription factors involved in apical meristem development and cell division and differentiation among others (Bartel, 2004; Carrington and Ambros, 2003). Several mRNA targets were beneath detection in total RNA, most likely due to their low abundance in vivo and/or their spatial regulation (Emery et al., 2003; Kidner and Martienssen, 2004; Palatnik et al., 2003). However, the expression of eleven validated mRNA targets was evident in poly(A)⁺ enriched RNA samples, extracted from wild-type Col-O and xrn4-5 inflorescence tissues (FIG. 5). These selected genes included ARF8 target of miR167, ARF10 and ARF17 targets of miR160, AP2-like target of miR172, a squamosa-promoter binding protein-like protein SPL10 target of miR156, MYB33 and MYB65 targets of miR159, HD-Zip transcription factor PHA VOLUTA (PHV) target of miR165, TCP2 and TCP4 targets of mir-JAW (miR319), and ARGONAUTE (AGO) target of miR168 (Bartel and Bartel, 2003; Park et al., 2002; Rhoades et al., 2002). Several transcripts, ARF10, ARF17, MYB33, MYB65 and PHV, showed an increase in accumulation of cleavage products corresponding to the 3′ end of the transcripts in the xrn4-5 mutant compared to the wild-type (FIG. 5A). For ARF8, AP2-like, TCP2 and TCP4, SPL10 and AGO transcripts, the abundance of the probable mRNA cleavage products was similar in inflorescence tissue harvested from the xrn4-5 plants compared to that of wild-type (FIG. 5B). Thus, inactivation of AtXRN4 impacted cleavage product accumulation for about half of the mRNA target mRNAs examined.

Discussion

In contrast to yeast, the in vivo function of XRNs in multicellular eukaryotes has not been extensively studied, mainly due to technical challenges associated with isolating knockout mutants to examine the effect on mRNA decay. As a result, prior to this work, there was no direct in vivo evidence that XRN enzymes function in mRNA decay in multicellular eukaryotes. Further, their role in mRNA/siRNA-triggered mRNA decay mechanisms was unknown. Using multiple insertional mutants, as well as complementation studies, we demonstrate that AtXRN4 is critical for the normal accumulation of several transcripts including AtFBL6, expressed protein 172F17 and several transcriptional factors validated as targets of mRNA-mediated cleavage. Interestingly, AtXRN4 inactivation leads to the stable accumulation of the 3′ end of these RNAs. This is striking because transcripts usually degrade without visible intermediates in multicellular eukaryotes and it has not been possible to engineer accumulation of intermediates (of nuclear encoded transcripts) with poly(G) tracts as in yeast (Kastenmayer and Green, 2000; Kastenmayer et al., 2001). In each case, the accumulation of the 3′ fragment is consistent with its increased stability in the absence of AtXRN4. The mRNA decay experiments provide the best evidence in the case of the 172F17 and SCARECROW-LIKE transcripts because the 3′ fragments are abundant enough and not too stable in the wild type to preclude valid comparisons (FIGS. 3 and 4A). For the SCARECROW-LIKE transcript, we and others have confirmed that the 3′ fragment, stabilized in the xrn4 mutants, is generated from mRNA cleavage and FIG. 5 demonstrates that AtXRN4 impacts a subset of other known mRNA targets and their 3′ cleavage products. This together with the compatibility of the 3′ fragments with AtXRN4 substrate requirements indicates that AtXRN4 degrades some of the intermediates resulting from mRNA and possibly siRNA-mediated cleavage.

Pioneering studies in yeast demonstrated the important role of Xrn1p as a general mRNA decay enzyme (Jacobs et al., 1998; Muhlrad et al., 1995), and this could be the case in multicellular eukaryotes, despite the relatively small number of transcripts that change abundance in the xrn4 mutant. Indeed, the recent application of microarray analysis to yeast xrn1Δ mutants was similar to ours in that it did not support a necessity of Xrn1p for global mRNA decay, presumably because the 3′ to 5′ decay pathway is sufficient for normal decay of most mRNAs (He et al., 2003). One could argue that the microarray studies are biased for the observation of polyadenylated mRNAs because the probes are oligo(dT) primed. However, at least for the unstable mRNAs of Arabidopsis that we sampled, mRNA decay rates monitored from total RNA samples were unaffected and consistent with the array data. Now that xrn4 knockouts are available, it should be possible to address whether diminishing the function of the 3′ to 5′ decay pathway in the cytoplasm is more deleterious when AtXRN4 is absent, similar to the situation in yeast.

It is logical that AtXRN4 has the greatest impact on transcripts that degrade, at least in part, via the generation of a 3′ intermediate because their decapping by cleavage makes them better substrates than the full-length capped mRNA for XRN but not the 3′ to 5′ pathway. The prominence of stable 3′ mRNA fragments among transcripts identified on the Arabidopsis arrays, and our mRNA decay experiments in the mutants, certainly indicate a role for AtXRN4 in their decay. FIGS. 4 and 5 indicate that some of these are generated from mRNA cleavage. Yet it is premature to assume that all arise from this mechanism because endonuclease cleavage resulting in visible intermediates is a known, albeit not prominent, mechanism for mRNA decay in eukaryotes (Binder et al., 1994; Chiba et al., 1999; Ross, 1996; Tourriere et al., 2002). Hybridization experiments have not yielded any evidence for mRNA or siRNAs that could trigger generation of the 3′ fragment for AtFBL6, although they may be extremely low abundance or tissue-specific. It is also relevant to note that two RNAs that could target cleavage of bromoprotein mRNA are present in a new database now containing about 1800 sequences of Arabidopsis 21 to 24 nt RNAs, and others may emerge as sequencing continues.

Nevertheless, it is the stabilization of the 3′ fragment of SCARECROW-LIKE transcripts which are well-known mRNA cleavage targets that prompts us to propose another step to the mRNA cleavage model as shown in FIG. 6. In this model, a given mRNA, such as miR171 in the case of SCARECROW-LIKE RNAs, acts to recruit a mRNA-associated RNP complex (which may be identical to RISC (Schwarz and Zamore, 2002)). Following recognition by sequence-specific base-pairing, the mRNA target is cleaved in or near a position corresponding to the middle of the mRNA, presumably by an endonuclease (Tang et al., 2003). The 5′ end of the transcript is then rapidly turned over, most likely by the exosome complex (van Hoof and Parker, 1999). Based on our data, we propose that AtXRN4 would simultaneously degrade the 3′ cleavage product in the 5′ to 3′ direction. AtXRN4 could also act downstream of the RISC complex in siRNA-mediated cleavage to help clean up 3′ intermediates resulting from post-transcriptional gene silencing or RNAi. Concurring with this model, the accumulation of additional cleavage products corresponding to validated mRNA target transcripts (ARF10, ARF17, MYB33, MYB65, and PHV) were also increased in xrn4-5 inflorescence tissues (FIG. 5A). Alternatively, in the absence of AtXRN4 (or as a result of a different regulatory influence, 3′ end cleavage fragments could be degraded via one or more alternative decay pathways (e.g. the mRNA targets in FIG. 5B). For example, some of the mRNA cleavage products in FIG. 5B that were unaffected in xrn4 mutants could be degraded by the exosome or alternative activity because their localization or temporal regulation (Kidner and Martienssen, 2004) differs from that of AtXRN4. Because some mRNA cleavage products may be produced in the nucleus, assessing the role of nuclear AtXRN2 and AtXRN3 (Kastenmayer and Green, 2000) in alternative pathways will be of particular interest. Finally at least one mRNA that targets the AP2-like transcripts in FIG. 5B, primarily downregulates its targets by translational repression rather than transcript cleavage (Aukerman and Sakai, 2003) similar to the situation in animals. If the accumulation of 3′ cleavage products is beneath detection in this scenario, then this would explain our findings for AP2-like transcripts (FIG. 5B).

Not all substrates of AtXRN4 produce stable 3′ end fragments. L19p transcripts shown in FIG. 3 appear representative of this class which is stabilized about 2-3 fold in the mutant without apparent intermediates. Several other transcripts found to be elevated in xrn4 from Table 1 seem to have these characteristics. At present it is unclear what makes these transcripts more dependent on AtXRN4 than the majority of transcripts. Perhaps they are in a cellular location or have a structure that is inaccessible by the exosome. Alternatively, they may be mainly expressed in specific cell types and some cell types may rely on the 5′ to 3′ pathway and the 3′ to 5′ pathway to differing extents. These transcripts should provide useful tools to address what sequences or other parameters make one mRNA a better substrate for one pathway compared to another or contribute to associations among pathways. In yeast, many of the relatively small subset of transcripts elevated in xrn1Δ mutants also were elevated in nonsense-mediated decay (NMD) mutants (He et al., 2003). Nonsense mediated decay proceeds 5′ to 3′ (Hagan et al., 1995; Muhlrad and Parker, 1994) and has been shown to occur in various eukaryotes including plants (Isshiki et al., 2001; Petracek et al., 2000). In the future, it should now be possible to use Arabidopsis to address if the NMD association of transcripts with high dependence on XRN function is of broad significance and help dissect its molecular basis.

The finding that AtXRN4 contributes to the decay of mRNA cleavage products in Arabidopsis is also likely to be of broad significance. Although most mRNAs are thought to repress translation in animal systems, some may work by cleavage and the cleavage products may be similarly elevated in xrn knockouts. Further, in plants and other systems exhibiting post-transcriptional gene silencing, some of the aberrant RNAs that likely help maintain silencing (Metzlaff et al., 1997), may also have free 5′ ends with 5′ monophosphates and thus be substrates of AtXRN4 or other XRN homologs. This could regulate the efficiency of silencing or RNAi mechanisms (Newbury and Woollard, 2004).

### REFERENCES

Aukerman, M. J., and Sakai, H. (2003). Regulation of flowering time and floral organ identity by a MicroRNA and its APETALA2-like target genes. Plant Cell 15, 2730-2741.
Bariola, P. A. et al. (1999). Regulation of S-like ribonuclease levels in Arabidopsis. Antisense inhibition of RNS1 or RNS2 elevates anthocyanin accumulation. Plant Physiol 119, 331-342.
Bartel, B., and Bartel, D. P. (2003). MicroRNAs: at the root of plant development? Plant Physiol 132, 709-717.
Bartel, D. P. (2004). MicroRNAs: genomics, biogenesis, mechanism, and function. Cell 116, 281-297.
Binder, R. et al. (1994). Evidence that the pathway of transferrin receptor mRNA degradation involves an endonucleolytic cleavage within the 3′ UTR and does not involve poly(A) tail shortening. Embo J 13, 1969-1980.
Carrington, J. C., and Ambros, V. (2003). Role of microRNAs in plant and animal development. Science 301, 336-338.
Cerutti, H. (2003). RNA interference: traveling in the cell and gaining functions? Trends Genet 19, 39-46.
Chiba, Y. et al. (1999). Evidence for autoregulation of cystathionine gamma-synthase mRNA stability in Arabidopsis. Science 286, 1371-1374.
Emery, J. F. et al. (2003). Radial patterning of Arabidopsis shoots by class III HD-ZIP and KANADI genes. Curr Biol 13, 1768-1774.
Gutierrez, R. A. et al. (2002). Identification of unstable transcripts in Arabidopsis by cDNA microarray analysis: rapid decay is associated with a group of touch- and specific clock-controlled genes. Proc Natl Acad Sci USA 99, 11513-11518.
Gutierrez, R. A. et al. (1999). Current perspectives on mRNA stability in plants: multiple levels and mechanisms of control. Trends Plant Sci 4, 429-438.
Hagan, K. W. et al. (1995). Characterization of cis-acting sequences and decay intermediates involved in nonsense-mediated mRNA turnover. Mol Cell Biol 15, 809-823.
Hannon, G. J. (2002). RNA interference. Nature 418, 244-251.
He, F. et al. (2003). Genome-wide analysis of mRNAs regulated by the nonsense-mediated and 5′ to 3′ mRNA decay pathways in yeast. Mol Cell 12, 1439-1452.
Higgs, D. C., and Colbert, J. T. (1994). Oat phytochrome A mRNA degradation appears to occur via two distinct pathways. Plant Cell 6, 1007-1019.
Isshiki, M. et al. (2001). Nonsense-mediated decay of mutant waxy mRNA in rice. Plant Physiol 125, 1388-1395.
Jacobs, J. S. et al. (1998). The 3′ to 5′ degradation of yeast mRNAs is a general mechanism for mRNA turnover that requires the SKI2 DEVH box protein and 3′ to 5′ exonucleases of the exosome complex. Embo J 17, 1497-1506.
Kasschau, K. D. et al. (2003). P1/HC-Pro, a viral suppressor of RNA silencing, interferes with Arabidopsis development and mRNA unction. Dev Cell 4, 205-217.
Kastenmayer, J. P., and Green, P. J. (2000). Novel features of the XRN-family in Arabidopsis: evidence that AtXRN4, one of several orthologs of nuclear Xrn2p/Rat1p, functions in the cytoplasm. Proc Natl Acad Sci USA 97, 13985-13990.
Kastenmayer, J. P. et al. (2001). Analysis of XRN orthologs by complementation of yeast mutants and localization of XRN-GFP fusion proteins. Methods Enzymol 342, 269-282.
Kidner, C. A., and Martienssen, R. A. (2004). Spatially restricted microRNA directs leaf polarity through ARGONAUTE1. Nature 428, 81-84.
Krysan, P. J. et al. (1999). T-DNA as an insertional mutagen in Arabidopsis. Plant Cell 11, 2283-2290.
Lejeune, F. et al. (2003). Nonsense-mediated mRNA decay in mammalian cells involves decapping, deadenylating, and exonucleolytic activities. Mol Cell 12, 675-687.
Llave, C. et al. (2002a). Endogenous and silencing-associated small RNAs in plants. Plant Cell 14, 1605-1619.
Llave, C. et al. (2002b). Cleavage of Scarecrow-like mRNA targets directed by a class of Arabidopsis mRNA. Science 297, 2053-2056.
Metzlaff, M. et al. (1997). RNA-mediated RNA degradation and chalcone synthase A silencing in petunia. Cell 88, 845-854.
Mourelatos, Z. et al. (2002). miRNPs: a novel class of ribonucleoproteins containing numerous microRNAs. Genes Dev 16, 720-728.
Muhlrad, D. et al. (1995). Turnover mechanisms of the stable yeast PGK1 mRNA. Mol Cell Biol 15, 2145-2156.
Muhlrad, D., and Parker, R. (1994). Premature translational termination triggers mRNA decapping. Nature 370, 578-581.
Newbury, S., and Woollard, A. (2004). The 5′-3′ exoribonuclease xrn-1 is essential for ventral epithelial enclosure during C. elegans embryogenesis. Rna 10, 59-65.
Newman, T. et al. (1994). Genes galore: a summary of methods for accessing results from large-scale partial sequencing of anonymous Arabidopsis cDNA clones. Plant Physiol 106, 1241-1255.
Newman, T. C. et al. (1993). DST sequences, highly conserved among plant SAUR genes, target reporter transcripts for rapid decay in tobacco. Plant Cell 5, 701-714.
Page, A. M. et al. (1998). Mutational analysis of exoribonuclease I from Saccharomyces cerevisiae. Nucleic Acids Res 26, 3707-3716.
Palatnik, J. F. et al. (2003). Control of leaf morphogenesis by microRNAs. Nature 425, 257-263.
Park, W. et al. (2002). CARPEL FACTORY, a Dicer homolog, and HEN1, a novel protein, act in microRNA metabolism in Arabidopsis thaliana. Curr Biol 12, 1484-1495.
Parker, R., and Song, H. (2004). The enzymes and control of eukaryotic mRNA turnover. Nat Struct Mol Biol 11, 121-127.
Perez-Amador, M. A. et al. (2001). New molecular phenotypes in the dst mutants of Arabidopsis revealed by DNA microarray analysis. Plant Cell 13, 2703-2717.
Petracek, M. E. et al. (2000). Premature termination codons destabilize ferredoxin-1 mRNA when ferredoxin-1 is translated. Plant J 21, 563-569.
Reinhart, B. J. et al. (2002). MicroRNAs in plants. Genes Dev 16, 1616-1626.
Rhoades, M. W., Reinhart, B. J., Lim, L. P., Burge, C. B., Bartel, B., and Bartel, D. P. (2002). Prediction of plant microRNA targets. Cell 110, 513-520.
Ross, J. (1996). Control of messenger RNA stability in higher eukaryotes. Trends Genet 12, 171-175.
Schwarz, D. S., and Zamore, P. D. (2002). Why do mRNAs live in the miRNP? Genes Dev 16, 1025-1031.
Sessions, A. et al. (2002). A high-throughput Arabidopsis reverse genetics system. Plant Cell 14, 2985-2994.
Sheth, U., and Parker, R. (2003). Decapping and decay of messenger RNA occur in cytoplasmic processing bodies. Science 300, 805-808.
Stevens, A. (1979). Evidence for a 5′ leads to 3′ direction of hydrolysis by a 5′ mononucleotide-producing exoribonuclease from Saccharomyces cerevisiae. Biochem Biophys Res Commun 86, 1126-1132.
Tang, G. et al. (2003). A biochemical framework for RNA silencing in plants. Genes Dev 17, 49-63.
Tanzer, M. M., and Meagher, R. B. (1995). Degradation of the soybean ribulose-1,5-bisphosphate carboxylase small-subunit mRNA, SRS4, initiates with endonucleolytic cleavage. Mol Cell Biol 15, 6641-6652.
Tourriere, H. et al. (2002). mRNA degradation machines in eukaryotic cells. Biochimie 84, 821-837.
van Hoof, A., and Parker, R. (1999). The exosome: a proteasome for RNA? Cell 99, 347-350.
Wang, Z., and Kiledjian, M. (2001). Functional link between the mammalian exosome and mRNA decapping. Cell 107, 751-762.
Wilusz, C. J. et al. (2001). The cap-to-tail guide to mRNA turnover. Nat Rev Mol Cell Biol 2, 237-246.

What is claimed is:

1. The use of mutant cells or non-human organisms in which the gene encoding XRN4 or its homologue is defective in a process for identifying RNA targeted for cleavage by mRNA or siRNA.

2. The use of claim 1 wherein said mutant non-human organism is a mutant Arabidopsis thaliana plant, wherein the gene encoding XRN4 is defective.

3. The use of claim 2 wherein said gene encoding XRN4 is disrupted by a T-DNA insert that renders the gene defective.

4. A method of identifying aberrant RNA or RNA targeted for cleavage by mRNA or siRNA comprising the steps of
optionally treating mutant cells or a non-human organism in which the gene encoding XRN4 or its homologue is defective with an agent that inhibits RNA synthesis;
isolating RNA from the cells or non-human organism; and
identifying aberrant RNA or RNA cleavage products in said isolated RNA by comparison with the wild type cells or non-human organism.

5. The method of claim 4, wherein the identifying step comprises the steps of
preparing microarray probes from said isolated RNA;
hybridizing said microarray probes with a microarray comprising DNA that represents gene transcripts from the same or similar type of cells or non-human organism;
detecting hybridization of the microarray probes;
relating changes in the hybridization signal of a probe with the identity of the corresponding genes represented on the microarray; and
characterizing the hybridized RNA as aberrant RNA or an RNA cleavage product by comparison with the wild-type cells or non-human organism or with known nucleic acid sequences selected from the group consisting of genes encoding mRNA, cDNA sequences, aberrant RNA sequences, mRNA sequences or siRNA sequences.

6. The method of claim 5 wherein said characterizing step comprises the use of Northern blot, Rapid Amplification of Complementary Ends (RACE), oligo-directed RnaseH cleavage, reverse transcriptase-polymerase chain reaction (RT-PCR) and/or DNA sequencing in characterizing said RNA as aberrant RNA or an RNA cleavage product.

7. The method of claim 6 wherein said mutant non-human organism is a mutant Arabidopsis thaliana plant, wherein the gene encoding XRN4 is defective.

8. The method of claim 7 wherein the gene encoding XRN4 is disrupted by a T-DNA insert that renders the gene defective.

9. The method of claim 4 wherein the identifying step comprises the steps of
preparing probes corresponding to at least one gene or other nucleic acid sequence of interest;
hybridizing said probes with a Northern blot that contains isolated RNA from the mutant cells or non-human organism and wild type RNA from the same or similar type of cells or non-human organism;
detecting hybridization of said probes with RNA on said Northern blot; and
characterizing said isolated RNA as aberrant RNA or an RNA cleavage product by comparison with the wild type cells or non-human organism or with known nucleic acid sequences selected from the group consisting of genes encoding mRNA, cDNA sequences, aberrant RNA sequences, mRNA sequences or siRNA sequences.

10. The method of claim 9 wherein said characterizing step comprises the use of Northern blot, oligo-directed RNaseH cleavage, Rapid Amplification of Complementary Ends (RACE), reverse transcriptase-polymerase chain reaction (RT-PCR) and/or DNA sequencing in characterizing said RNA as aberrant RNA or an RNA cleavage product.

11. The method of claim 9 wherein said mutant non-human organism is a mutant Arabidopsis thaliana plant wherein the gene encoding XRN4 is defective.

12. The method of claim 11 wherein the gene encoding XRN4 is disrupted by a T-DNA insert that renders the gene defective.
